(19) United States
(12) Patent Application Publication
(10) Pub. No.: US 2015/0099807 A1
(43) Pub. Date: Apr. 9, 2015

(54) PRESERVATIVE FREE BIMATOPROST SOLUTIONS

(71) Applicant: ALLERGAN, INC., IRVINE, CA (US)

(72) Inventors: Sukhon Likitlersuang, Irvine, CA (US); Ajay Parashar, Irvine, CA (US); Chetan P. Pujara, Irvine, CA (US); William F. Kelly, Rancho Santa Margarita, CA (US)

(21) Appl. No.: 14/308,320

(22) Filed: Jun. 18, 2014

Related U.S. Application Data
(63) Continuation of application No. 13/812,594, filed on May 22, 2013, now abandoned.
(60) Provisional application No. 61/368,688, filed on Jul. 29, 2010.

(51) Int. Cl. A61K 31/5575

(52) U.S. Cl. CPC A61K 31/5575
USPC 514/622

(57) ABSTRACT

The present invention is directed to preservative-free solutions of bimatoprost for lowering intraocular pressure and treatment of glaucoma.

### CROSS REFERENCE TO RELATED APPLICATIONS

This Application is a continuation of U.S. patent application Ser. No. 13/812,594, filed Jan. 28, 2013, which is a national phase application under 35 U.S.C. §371 of PCT Patent Application No. PCT/US11/45652, which claims priority to U.S. Provisional Patent Application Ser. No. 61/368,688, which was filed on Jul. 29, 2010, both of which are incorporated herein by reference in their entirety.

### FIELD OF THE INVENTION

The present application is directed to preservative-free formulations of bimatoprost.

### BACKGROUND OF THE INVENTION

Bimatoprost is a prostamide, a synthetic analog of prostaglandin F_{2α} (PGF_{2α}) with potent ocular hypotensive activity. Bimatoprost lowers intraocular pressure (IOP) in patients with glaucoma or ocular hypertension by increasing outflow of aqueous humor through both the trabecular meshwork and uveoscleral routes.

Use of preservative containing eye drops has been implicated in the development or worsening of ocular surface disease. Management of open angle glaucoma and ocular hypertension require long term treatment with eye drops containing preservatives. Symptoms and signs of ocular surface disease such as ocular surface breakdown, irritation, burning, foreign body sensation, dryness, inadequate quantity of tears etc. are prevalent in a large proportion of patients with open angle glaucoma and ocular hypertension.

Compared to eye drops preserved with benzalkonium chloride, preservative-free eye drops induce significantly fewer ocular symptoms and signs of irritation in patients, such as pain or discomfort, foreign body sensation, stinging or burning, and dry eye sensation.

Patients experiencing hypersensitivity reactions with benzalkonium chloride cannot use the commercial bimatoprost product containing benzalkonium chloride such as LUMIGAN which is preserved with 0.005% w/v benzalkonium chloride. Benzalkonium chloride also may be absorbed by the soft contact lenses therefore patients wearing soft contact lenses are advised to remove lenses prior to administration and wait at least 15 minutes before reinserting them.

### SUMMARY OF THE INVENTION

The present invention is directed to bimatoprost formulations (e.g., solutions) without benzalkonium chloride which are superior from a safety, tolerability and patient compliance standpoint while maintaining and/or improving its efficacy of TOP lowering and be available for use by patients hypersensitive to benzalkonium chloride and be convenient for patients wearing soft contact lenses.

Bimatoprost ophthalmic solution without preservative is a clear, isotonic, sterile solution. The drug product contains bimatoprost as the active ingredient. The inactive ingredients are tonicity and buffer agents, and purified water. Suitable buffers such as sodium phosphate dibasic heptahydrate and citric acid monohydrate and suitable tonicity agents such as sodium chloride may be included. The final solution would be an aqueous solution having a pH value within the range of about 7 to 8, preferably 7.3 and osmolality in range of 280-370 mOsmol/kg.

The present invention can be made generally according to the teachings of U.S. Pat. No. 5,688,819, which is hereby incorporated by reference in its entirety.

Some of the embodiments of the present invention are as follows:

1) A preservative free bimatoprost composition for lowering intraocular pressure in a patient comprising the following formulation: about 0.03% w/v bimatoprost; about 0.268% w/v sodium phosphate heptahydrate; about 0.014% w/v citric acid monohydrate; about 0.83% w/v sodium chloride; water and having a pH of about 7.3;
2) A preservative free bimatoprost composition for lowering intraocular pressure in a human patient comprising the following formulation: 0.03% w/v bimatoprost; 0.268% w/v sodium phosphate heptahydrate; 0.014% w/v citric acid monohydrate; 0.83% w/v sodium chloride; water, hydrochloric acid, sodium hydroxide and having a pH of about 7.3;
3) The bimatoprost composition of paragraphs 1 and 2 wherein the composition is a solution and is useful for treating glaucoma;
4) The bimatoprost composition of paragraph 3 wherein the solution is contained in a unit dose kit form;
5) The bimatoprost composition of paragraphs 1-4 wherein the composition is a solution and is applied once a day to each eye;
6) The bimatoprost solution of paragraphs 1-4 wherein the composition is a solution and is applied twice a day to each eye;
7) The bimatoprost composition of paragraph 1 wherein the composition is a solution and has greater bioavailability of bimatoprost in the eye of the patient with fewer side-effects than bimatoprost preserved with benzalkonium chloride;
8) The composition of paragraph 1 wherein the composition may be a solution, emulsion, dispersion, suspension, reverse emulsion and microemulsion;
9) The composition of paragraph 1 wherein the composition is contained in a unit-dose vial.
10) The composition of paragraph 1 wherein the composition is contained in a multi-dose vial which has anti-preservative properties such as metal-ions imbedded in its dispensing tip; and,
11) The composition of paragraph 12 wherein the metal ions are silver ions.

### DETAILED DESCRIPTION OF THE INVENTION

One bimatoprost ophthalmic formulation of the present invention without preservative is shown in Table-1.

TABLE 1
Example of a bimatoprost ophthalmic solution without
preservative according to the present invention:

| Ingredients | Units | Grade | Amount |
|---|---|---|---|
| Bimatoprost | % w/v | N/A | 0.03 |
| Sodium Phosphate Dibasic | % w/v | USP | 0.268 |
| Heptahydrate | | | |
| Citric Acid Monohydrate | % w/v | USP/Ph Eur | 0.014 |
| Sodium Chloride | % w/v | USP/Ph Eur | 0.83 |
| Hydrochloric Acid | % w/v | USP/Ph Eur | pH 7.3 |
| Sodium Hydroxide | % w/v | USP/Ph Eur | pH 7.3 |
| Purified Water/WFI | Q.S. | USP/Ph Eur | QS |

The present invention is directed to the same bimatoprost formulation as commercially available LUMIGAN 0.03 but without benzalkonium chloride as a preservative and in unit-dose or multi-dose form. As a result of the removal of benzalkonium chloride, the present invention results in greater bioavailability of the active ingredient bimatoprost in the eye without the unwanted side-effects associated with the preservative benzalkonium chloride such as hyperemia. This results in a formulation with the same or improved efficacy of the product in lowering TOP per dosage unit, fewer side-effects and superior patient compliance. Other side effects which may be avoided include visual disturbance, ocular burning, foreign body sensation, eye pain, blepharitis, cataract, superficial punctate keratitis, eyelid erythema, ocular irritation, eye discharge, tearing, photophobia, allergic conjunctivitis, asthenopia, conjunctival edema, conjunctival hemorrhage, and intraocular inflammation.

1. A preservative free bimatoprost composition for lowering intraocular pressure in a patient comprising the following formulation: about 0.03% w/v bimatoprost; about 0.268% w/v sodium phosphate heptahydrate; about 0.014% w/v citric acid monohydrate; about 0.83% w/v sodium chloride; water and having a pH of about 7.3.

2. A preservative free bimatoprost composition for lowering intraocular pressure in a human patient comprising the following formulation: 0.03% w/v bimatoprost; 0.268% w/v sodium phosphate heptahydrate; 0.014% w/v citric acid monohydrate; 0.83% w/v sodium chloride; water, hydrochloric acid, sodium hydroxide and having a pH of about 7.3.

3. The bimatoprost composition of claim 1 wherein the composition is a solution and is useful for treating glaucoma.

4. The bimatoprost composition of claim 3 wherein the solution is contained in a unit dose kit form.

5. The bimatoprost composition of claim 1 wherein the composition is a solution and is applied once a day to each eye.

6. The bimatoprost solution of claim 2 wherein the composition is a solution and is applied twice a day to each eye.

7. The bimatoprost composition of claim 1 wherein the composition is a solution and has greater bioavailability of bimatoprost in the eye of the patient with fewer side-effects than bimatoprost preserved with benzalkonium chloride.
